(19) United States
(12) Patent Application Publication
(10) Pub. No.: US 2009/0305436 A1
(43) Pub. Date: Dec. 10, 2009

(54) DEVICE AND METHOD FOR DETECTION OF A PREGNANCY ASSOCIATED HORMONE

(75) Inventors: Christopher Plummer, Herefordshire (GB); Jeffrey Bauer, Carlsbad, CA (US)

Correspondence Address: BIRCH STEWART KOLASCH & BIRCH, PO BOX 747, FALLS CHURCH, VA, 22040-0747, US

(73) Assignee: British Pregnancy Adivsory Service, Warwickshire (GB)

(21) Appl. No.: 12/297,434

(22) PCT Filed: Apr. 20, 2007
(86) PCT No.: PCT/GB2007/001454

§ 371 Date: Feb. 24, 2009

(30) Foreign Application Priority Data
Apr. 21, 2006 (GB) 0607964.4
May 17, 2006 (GB) 0609798.4

(51) Int. Cl. G01N 33/76; G01N 33/53

(52) U.S. Cl. 436/510; 422/56

(57) ABSTRACT

The invention provides a device (10) for detection and providing an indication of the concentration of a single pregnancy associated hormone (especially hCG), comprising a matrix (16) defining an axial flow path, the matrix comprising: (i) a sample receiving zone (18) at the upstream end of the flow path, for receiving a fluid sample; (ii) a plurality of test zones (22) positioned within the flow path and downstream from the sample receiving zone (18), wherein each test zone is capable of detecting a different concentration of hormone compared with an adjacent test zone; and (iii) an indicator (26) for recording a previous hormone concentration recorded by the same or a previous device. The devices are especially useful to give an indication of a miscarriage or non-natural termination. Methods of using the assay devices are also provided.

The invention relates to assay kits for monitoring the concentration of pregnancy hormones such as chorionic gonadotrophin, especially human chorionic gonadotrophin (hCG), in samples obtained from pregnant female mammals, such as humans. The assays are utilised to monitor the early stages of pregnancy, to provide an early indication of abnormal pregnancies and normal abortions, and may also provide an indication of whether a chemically-induced termination has been successful.

Human chorionic gonadotrophin (hCG) is a protein hormone that stimulates progesterone secretion. For over 25 years, researchers have investigated the molecular forms of hCG in pregnancy and in the number of different physiopathological contexts, such as trophoblastic diseases, testicular cancers and a number of tumours. hCG is found in a number of forms, including intact hCG, free-α subunit, free-β subunit and various fragments and isoforms of the hormone.

hCG has two non-covalently associated subunits (α and β) that share about 10% sequence identity and fall into similar conformations. The α and β subunits have 5 and 6 disulphide bridges respectively.

The α subunit has 92 amino acids and is expressed by both the pituitary gland and placental tissue. The β subunit (145 amino acids) determines the biological and immunoreactive uniqueness of the intact hCG molecule. Antibodies developed against the C-terminal peptide have proved to be especially useful in the detection of hCG. Such antibodies demonstrate minimal, or no cross reactivity, with luteinising hormone (LH). Such C-terminal antibodies have proven especially useful in measuring low hCG concentrations during the early stages of pregnancy.

The detection of hCG, and indeed another hormone, progesterone, in the detection of pregnancies has been known for many years. Such test devices include dipsticks such as EP0125118, membrane assays such as U.S. Pat. No. 4,818,677, and lateral flow assay systems such as GB2,204,398 and EP0306772. Most of these devices provide a qualitative assay. That is, they determine whether hCG has reached above a certain concentration and provide a simple, “yes/no” answer of whether the woman is pregnant.

Typically, the woman then has to wait for some 10-12 weeks before a scan is carried out to be able to determine whether the pregnancy is normal and has been successful.

The inventors have realised that there is a need to provide a simple, easy to use and cost-effective way of allowing the woman to confirm that she still is pregnant. Such an assay also allows the early detection of problems with the pregnancy and naturally-occurring abortions. Often such abortions are missed early on in the pregnancy, resulting in considerable disappointment and distress later in the pregnancy when the first scan is carried out. Moreover, the inventors have realised that such test kits can be extended to provide a simple way of monitoring chemically-induced terminations.

U.S. Pat. No. 5,786,220 discloses a method for distinguishing between a normal and an abnormal pregnancy. The method relies on the determination of the concentration of progesterone or a progesterone metabolite. A concentration above a pre-determined threshold is indicated as indicating a normal pregnancy. The concentration of hCG may be measured in conjunction with the progesterone concentration. Visible label is accumulated when hCG exceeds a threshold level, but absent below a value. The hCG is indicated as being used to avoid false prediction of normal pregnancy, when in fact the individual was not pregnant at the time of testing. The preferred assay disclosed provides two signals, wherein the presence of one signal and absence of another signal indicates a normal pregnancy. Thresholds of 5 ng/ml to 50 ng/ml of blood, plasma or serum of progesterone are discussed. 25 mIU/ml to 100 mIU/ml are the threshold levels for hCG.

The patent notes in passing that a serial rise of hCG from day-to-day may be used to indicate viable pregnancy when progesterone levels remain above 25 ng/ml. An hCG level of below 1000 mIU/ml eight weeks after conception, in conjunction with 25 ng/ml progesterone is stated to indicate that the pregnancy is at risk. A semi-quantitative assay for measuring hCG and progesterone in serum is described. Five concentrations of hCG are suggested: 500, 1000, 1500, 2000 and 2500 mIU. The final line only shows colour when 2500 mIU of hCG is above 2500 mIU. Progesterone is measured in the same assay.

US2005/0130120 discloses assays for detecting and differentiating different analytes in a single fluid sample. The aim is to measure multiple analytes in a sample, such as hormones, metabolites, and antigens from infectious agents, such as bacteria and viruses.

US2003/1075991A discloses an optical based lateral flow matrix for quantifying hCG from 0-150,000 mIU/ml in a sample. The technique uses a capture zone immobilised with anti-hCG probes. The concentration of hCG captured is determined using a complex optical beam arrangement. It is used to assist in the prognosis of early pregnancy.

US2003/0003597 discloses an alternative assay for the determination of pregnancy outcome. The assay looks at the specific very early pregnancy isoform of hCG in a sample. This is compared to a normalised amount in normal pregnant subjects or from non-pregnant subjects.

EP 1571451A discloses taking a sample from a female subject and measuring hCG level in the sample. From the sample they are able to extrapolate to identify an estimated due date. One feature of the method disclosed is to take a number of samples to estimate the expected delivery date or date of conception. The method looks at the increase in hCG levels and matches this to a predefined concentration curve of hCG to identify the dates. A complex device is proposed comprising a microprocessor to allow hCG tests to be carried out on a number of days and data recorded. The microprocessor is usually in a separate reader to the assay device used to detect the analyte.

In contrast, the Applicants propose a simple, inexpensive device to allow a decrease in hCG levels to be readily detected by a previously pregnant woman.

Barnhart K., et al., Obstetrics and Gynaecology (2004), Vol. 104, pages 50-54 and 975-981, discusses the rise of hCG in pregnant women and decline of serum hCG after spontaneous abortions. This indicated a rate of hCG decrease in spontaneous abortions and is described by a quadratic profile with a faster decline of the hCG with higher presentation levels. The rate of decline ranged from 21% to 35% at 2 days and 60% to 84% at 7 days. A rate of decline of less than 21% at 2 days or 60% at 7 days suggested retained trophoblasts or ectopic pregnancy. Hence the drop in hCG levels can be used to indicate the abortion of a fetus or other problems with a pregnancy.

In “Improving Medical Abortion”, Christian Fiala, Dept. of Woman and Child Health, Karolinska Inst., Stockholm, Sweden, 2005, ISBN 91-7140-458-9, it is suggested that a further research aim of the group is to identify methods of allowing women to verify successful abortion using a urinary hCG test. This shows that such tests were not currently available. It also confirms that hCG levels drop to 20% pre-abortion levels, one week after abortion.

The inventors have realised that this may be used to monitor chemically induced terminations. Such terminations, often known as “the abortion pill” are available in many countries in the early weeks of pregnancy (the first 9 weeks in the UK). One method commonly used utilises a drug called mifepristone (also known as Mifegyne or RU486). This is usually given at a clinic. Patients are then usually sent home. After a period of up to two days a second drug, prostaglandin, is given. One problem associated with such methods is that a small proportion of the treatments may not be effective. There is therefore a need to have an early indication that the treatment has not been effective so that further treatment and a diagnosis then be sought from a doctor and can be carried out within the legal time-limits set for such treatments. The ability to do this at home, by the patient, without a visit to a clinic, allows this to be carried out discretely by the patient, reducing what is already often a traumatic time for them.

Measuring the hCG levels before the treatment also provides a baseline measurement. The concentration of hCG is expected to provide an early indication of when the termination could occur.

The inventors have realised that there is a need for a simple, easy-to-use test kit that can be used at home. It preferably utilises and adapts conventional and well-proven technology in order to produce a cost-effective home use assay kit. It may be used as comfort, to monitor the gradual increase in hCG with a normal pregnancy, or more preferably to monitor natural or non-natural abortions.

Accordingly, a first aspect of the invention provides a device for detection and providing an indication of a quantity of a single pregnancy associated hormone in a sample comprising a matrix defining an axial flow path, the matrix comprising:

(i) A sample receiving zone at the up-stream end of the flow path, for receiving a fluid sample; and
(ii) A plurality of test zones positioned within the flow path and downstream from the sample receiving zone, wherein each test zone is capable of detecting a different concentration of hormone compared with an adjacent test zone; additionally comprising:
(iii) An indicator, for example, on the device, or packaging for the device, for recording a hormone concentration recorded by the same or a different type of device.

The inventors realised that when their device is being used in a home it is often possible for the users to lose any pieces of paper on which they might have written down the concentration recorded by the initially used device. Hence, they have realised that it would be useful for the device to have an indicator for recording a previous hormone concentration assay on a later used device for an immediate ready comparison. This has the benefit of reducing the likelihood of previous test results being lost.

Accordingly, the device comprises an indicator for recording a hormone concentration recorded by the same or a different device. This indictor may simply be a position on which a mark at the appropriate concentration on the device may be placed. This mark may be placed upon the device by a pen. Alternatively, for example, the device may comprise a series of raised portions moulded in a casing of the device. The raised portions may be deformable so that the appropriate concentration can simply be pressed in to cause a depression against the appropriate concentration. The indicator may be fixed, or detachable to allow it to be transferred to another device. It may also be on the packaging of the device to allow a previous concentration to be recorded. It may comprise a number or colour coded strip to read and record levels. The indicator may optionally contain an indicator to show that an hCG level has been reached where ultrasound imaging may be successfully carried out.

The packaging may be a box or other container or wrapping in which the device is packaged before use.

Preferably the device is disposable. The term disposable indicates that the device is a single use device. Most preferably an indication of a quantity of the hormone is capable of being directly obtained by a user of the device by visually inspecting the device (for example, without having to use a separate reader).

Preferably, the device is adapted to detect a decrease in the pregnancy associated hormone associated with a miscarriage or non-naturally termination of a pregnancy. As described below, the Applicants have optimised the device to detect such occurrences.

The test kit is preferably adapted to detect the natural increase in hCG levels as pregnancy progresses (to give a comfort factor to mothers before, e.g. ultrasound can be carried out) and also to be able to detect the rapid falling off the concentration of hCG when the fetus is aborted.

The device is preferably in the form of a dipstick. However, the assay may also be used in the form of other lateral flow devices generally known in the art. For example, the lateral flow device may comprise a well into which a sample is placed, for example via a pipette or syringe. Fluid from the sample is then drawn into the matrix and through the device.

It is preferred that a single pregnancy associated hormone is detected. The pregnancy associated hormone is most preferably chorionic gonadotrophin. However, progesterone may also be used. Preferably, the pregnancy associated hormone is a human hormone. However, it is envisaged that the hormone to be detected may be from any mammal, for example to allow the early progression of pregnancies in commercially important animals, such as race horses, or alternatively to allow the early pregnancy in rare mammals to be followed.

The matrix may be any conventional solid phase of a type generally used in performing immunoassays, including dipsticks, membranes, absorptive pads, beads, micro titre wells, test tubes and the like. As indicated previously, such devices are generally known in the art. Typical matrix materials include high density polyethylene sheet material, paper, nitrocellulose, derivatised nylon, cellulose, and the like known in the art. These may optionally be blocked with, for example, whole or derivatised bovine serum albumin, whole animal serum, casein or non-fat dry milk.

The sample receiving zone is a portion of the matrix to which the individual sample is applied. The sample may be, for example, blood, serum or urine. The sample receiving zone may remove blood cells, such as red blood cells, white blood cells and/or different hormones from the sample by, for example, utilising immobilised antibodies to the cells or hormones to be removed. Such systems are generally known in the art. Preferably, urine is the sample.

The plurality of test zones positioned within the flow path and downstream from the sample receiving zone detect different concentrations of hormone compared with each other. Preferably, these provide a visual indication of the concentration. In its simplest form, this may appear as a colour in a window within the device against a representation, figure or numeral indicating a particular concentration or level of hormone. Alternatively, more complex displays, such as liquid crystal displays, may be used to give a visual indication of the level of hormone. Devices for converting hCG binding into an electrical or LCD presentation are known per se. However, such electrically based systems increase the cost of the device. In a preferred form the display may be just a series of coloured test zones. The greater the number of coloured test zones, the greater concentration of hormone. The test zones that become coloured may then be recorded on the indicator for comparison with a later assay.

Preferably, the test zones each contain restraint elements which permit the restraint of labelled pregnancy associated hormone. The restraint elements will usually be antibodies or fragments of antibodies. Preferably, these antibodies will be specific for the pregnancy associated hormone. That is, they bind the hormone to be analysed and not substantially other hormones or other compounds within the sample.

Typically, the restraint elements will be immobilised within the test zone.

One potential problem identified by the inventor has been non-specific binding of the labelled reagent at lower concentrations of hormone. They have realised that this may be reduced using an avidin-biotin system.

Avidin (and streptavidin) is a known protein originally found isolated from egg white that binds biotin with an affinity constant of 10⁻¹⁵. The inventor has realised that the sensitivity of the assay may be improved by utilising avidin/biotin binding.

Preferably the restraint element comprises a first half of a avidin/biotin binding pair (that is, biotin or avidin) and the device comprises an anti-pregnancy associated hormone antibody (such as an anti-hCG antibody) linked to a second half of an avidin/biotin binding pair (that is avidin or biotin). The antibody may bind to the hormone and is in turn is captured and bound to the restraint element in the test zone via the binding of the avidin/biotin pair. The antibody may be provided at the sample receiving zone or a label zone (described below) and bind to the hormone in solution, prior to being trapped at the test zone via interaction between the avidin/biotin binding pair.

The term “avidin” is intended to include both avidin and streptavidin (originally isolated from bacteria). Methods of immobilising avidin and biotin on matrixes are generally known in the art, as are methods of attaching avidin or biotin to proteins, such as antibodies. The avidinylated or biotinylated antibody is preferably a monoclonal anti-hCG antibody.

Preferably the labelled reagent is provided as a labelled anti-pregnancy associated hormone (such as anti-hCG) polyclonal antibody. This is preferably in addition to the antibody attached to a second half of the avidin/biotin pair.

Antibodies are polypeptides substantially encoded by an immunoglobulin gene or immunoglobulin genes or fragments thereof. The antibody may be from any one of the immunoglobulin classes, IgG, IgM, IgA, IgD or IgE. The antibody may be polyclonal or monoclonal. Antibodies may include a complete immunoglobulin or fragments thereof. Fragments thereof include Fab, Fv and F(ab′)2, Fab′, and the like. Antibodies may also include chimeric antibodies and fragments thereof made by recombinant methods. Suitable antibodies are well known in the art.

Preferably, the concentration of pregnancy associated hormone detected increases between a test zone closest to the sample receiving zone and a test zone further away from, and downstream of, the sample receiving zone. That is, lower concentrations of hormone are detected closer to the sample receiving zone, whilst higher concentrations of pregnancy associated hormone are detected further away from, and downstream of, the sample receiving zone.

Preferably, the matrix is generally linear and the test zones may be provided in portions of the matrix extending out of the plane of the matrix. Sample running through the matrix may be diverted, for example, via capillary action through a portion of matrix extending out of the general plane of the matrix, into the test zone where it meets, for example, the restraint elements. This helps to prevent leaching of, for example, the dyes for detecting hCG, between each test zone. A molecular valve as may be used to direct sample in a one-way direction into the test zone may be provided.

The sample preferably flows from the sample receiving zone, optionally through the label zone, and through the lower concentration test zones and to the higher concentration test zones.

Each separate test zone detects a different concentration of hormone. This may be achieved by simply providing different concentrations of antibodies, for example by serial dilution of antibodies, at each test zone. Alternatively, different concentrations may be detected because of the ability of, for example, hCG to pass through to a zone further away from the sample receiving zone. Each test zone will attract labelled hCG. Progressively higher amounts of labelled hCG will be able to pass to adjacent test zones.

The antibodies are preferably immobilised on each test zone.

For example, the test zones may comprise detection zones for 400, 15000 and 40000 mIU/ml of hormone. If the sample only contains about 400 mIU/ml of hormone, then substantially only the test zone with 400 mIU/ml will become labelled. However, if the sample contains above 40000 mIU/ml of hormone, then test zones for 400, 15000 and 40000 mIU/ml may become labelled.

Preferably a label zone is provided within the flow path and between the sample receiving zone and plurality of test zones, said label zone comprising a labelled reagent which is capable of binding the pregnancy associated hormone and is mobilisable in the presence of a fluid sample, wherein said test zones each contain restraint elements which permit the restraint of labelled pregnancy associated hormone. The label zone may be the same part of the device as the sample receiving zone.

The labelled reagent is preferably a detectable label attached to a specific binding member such as an antibody capable of binding to the pregnancy associated hormone. The labelled reagent is released by fluid from the sample passing through the label zone. The labelled reagent then binds to the pregnancy associated hormone. The attachment may be covalent or non-covalent binding. Such labels are themselves well-known in the art. The label need not be specific for only the pregnancy associated hormone, as other substances labelled with the labelled reagent may simply wash through the test zones, with the labelled pregnancy associated hormone being immobilised by the restraint elements that are preferably provided in each test zone. However, preferably the labelled reagent preferably comprises a binding member such as an antibody or fragment of an antibody that is specific for the pregnancy associated hormone. The antibody, or fragment thereof, or indeed an alternative binding member, is labelled. The label allows the labelled reagent to produce a detectable signal that is related to the presence of the pregnancy related hormone in the sample. The label itself may form the labelled reagent.

The label may be any substance which is capable of producing a visually detectable signal, or indeed a signal detectably by instrumental means. Labels include enzymes and substrates, chromogens, fluorescent compounds and radioactive labels. Other suitable labels include latex particles or beads, colloidal metal particles such as gold, colloidal non-metallic particles such as selenium or tellurium or other such labels generally well-known in this field. Preferably, the label produces a coloured signal that is detectable visually with the eye, without the need for further instrumentation.

Preferably, the device comprises between 6 and 30 separate test zones, most preferably between 6 and 20, especially 8-15, or 10-15, most preferably 9 or 12 separate test zones. Each of those test zones preferably has a different concentration of hormone that it can detect.

Preferably the hormone to be detected is hCG. Preferably the device can detect a range of hCG between 10-260,000 mIU/ml of sample. Most preferably the concentration of hCG detectable is between 40-165,000 mIU/ml.

The hCG detected is preferably total hCG, and is preferably not limited to a single particular isoform of hCG.

The inventors have looked at the results of empirical studies reported on the following web sites: americanpregnancy.org, ivfer.com, greenjournal.org, spals.com, birth.com.au, sydpath.stvincents.com.au, Human Reproduction, http://humrep.oxfordjournals.org/cgi/reprint/14/9/2375, ivfer.com/hcg_survey.htm and fertilityplus.org/faq/hpt.html. They compared and averaged the data of hCG observed over the course of early pregnancy and have used that to model the most preferred arrangements of the concentrations of hormone to be detected. The results that they have identified are shown below.

| | Days | | | | | | | | | | | | | | |
|---|---|---|---|---|---|---|---|---|---|---|---|---|---|---|---|
| | 0 | 3 | 6 | 9 | 12 | 15 | 18 | 21 | 24 | 27 | 30 | 36 | 42 | 69 | 99 |
| Average | 3 | | 10 | 25 | 44 | 189 | 734.6 | 2833.7 | 4392.3 | 8062.5 | 21193.33 | 76375 | 114883.3 | 154987.5 | 114983.3 |
| all | | | | | | | | | | | | | | | |
| Rate of | | | | | 1.8 | 4.3 | 3.7 | 3.9 | 1.5 | 1.8 | 2.6 | 3.6 | 1.5 | 1.3 | |
| change | | | | | | | | | | | | | | | |

Figures indicate mIU/ml of hCG.

One matter to note from this table is that between days 15 and 18 the inventors have identified a rise that is substantially higher than would normally have been expected. This surge in hCG levels occurs at the point at which ovulation would normally occur and lasting for about 10 days. This is consistent with reported physiological symptoms by pregnant women. It will be noted that there is an increase between approximately 189 mIU/ml to approximately 735 mIU/ml on days 15 and 18, and then continuing to rise to over 2000 on day 21. The importance of this observation means that concentrations of between 500-1500, more preferably 600-1400, 700-1300, 800-1200, 900-1100 mIU/ml of hCG may not need to be detected by the device. This reduces the number of separate test zones required, or alternatively means that additional test zones can be used to improve the range of hCG detected.

Preferably the test kit is capable of detecting a concentrations of hCG at approximately 1500-2500 mIU/ml. This is the typical concentration at which pregnancy is sufficiently progressed so that pregnancy can be confirmed using an ultrasound scan via a vaginal probe. A concentration of around 4000-7000 mIU/ml may additionally or alternatively be detected. 4000-7000 mIU/ml indicates the typical earliest point where ultrasound scanning with an abdominal probe may be used to confirm pregnancy.

An additional factor to take into account for identifying the preferred concentrations of hCG to be detected is the typical stage at which early medical abortions are carried out, for example by clinical termination. Typically, these occur at 35 days gestation where hCG levels are of the order of 120,000 mIU/ml. The latest point for early medical abortion in the UK is currently 63 days gestation. Ultrasound can be used for detecting pregnancy from about 5 weeks when hCG levels could be as high as 150,000 mIU/ml. If a fetus is aborted, either naturally or via artificial means, there is a rapid decrease in the amount of hCG observed.

The test kit therefore preferably should be able to detect the natural increase in hCG levels as the pregnancy progresses (therefore giving a comfort factor to expectant mothers who would like confirmation that the pregnancy is proceeding well) and also be able to detect the rapid falling off of the concentration of hCG when the fetus is aborted.

Preferably, the concentrations of hormone to be detected in each of the test zones may be: 40, 100, 400, 1600, 4000, 8000, 16000, 32000, 64000, 96000, 132000, 164000 mIU/ml, or most preferably 2500, 7000, 15000, 27500, 40000, 55000, 75000, 100000 mIU/ml. The latter concentrations provide especially advantageous coverage for the device using fewer concentrations.

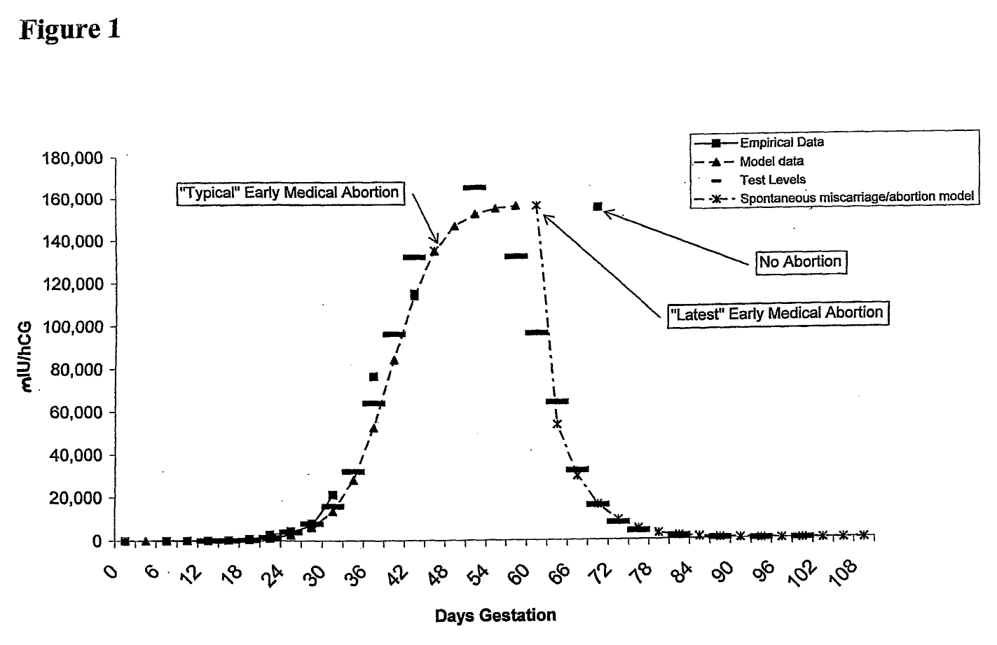

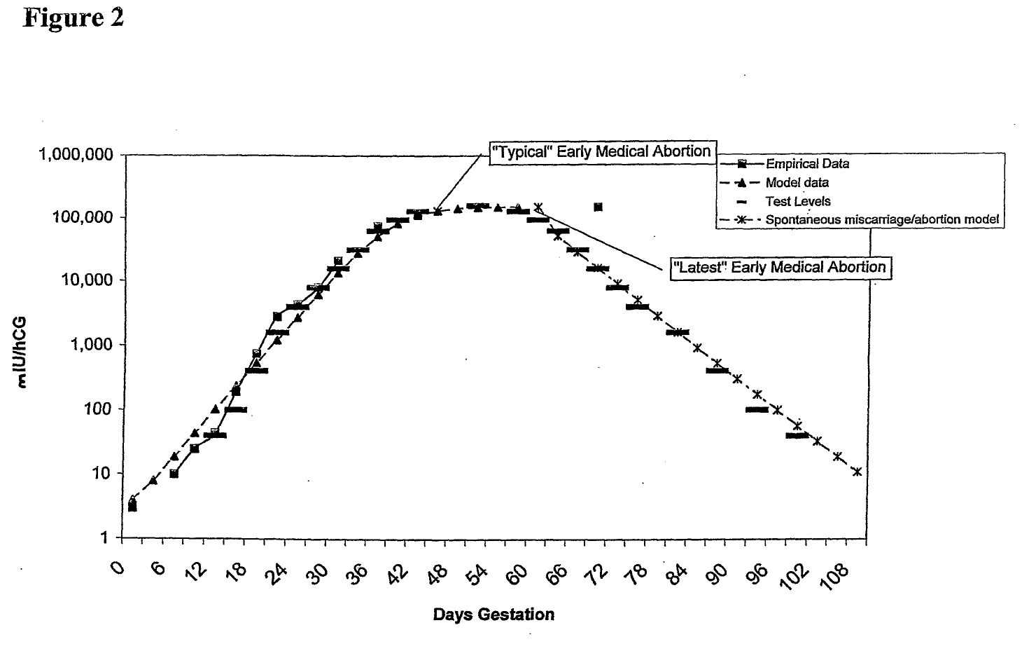

FIG. 1 shows the rise and fall of hCG levels through normal pregnancy and following a spontaneous miscarriage or termination. This data is also shown in FIG. 2 on a logarithmic scale. The preferred concentrations of hCG to be detected by the device (“test levels”) have been superimposed. This illustrates the ability of the test device to detect both normal pregnancy associated increases, as well as decrease in hCG due to miscarriage/abortion. The concentration of hCG was determined using data from a number of publicly available sources, as discussed above.

The “model data” is a best fit line of the available empirical data.

Where the assay is used to detect an induced termination, the assay is typically used just before the termination to determine a base line. The assay is repeated, typically using separate devices, on day 4 and, if the results indicate a need for it, day 8, in order to follow the decrease in concentration of hCG.

Preferably, the devices of the invention additionally comprise a control zone position within a flow path and downstream of the control means and comprise a means permitting the indication of the completion of the assay. Such control zones are well known in the devices generally known in the art. For example, it may simply be a chemical that becomes visible when moisture from the sample reaches that point on the device.

Preferably, the device is a dipstick. In such a situation it may comprise a wick forming at least a portion of the sample receiving zone for the uptake of a sample, for example from a urine sample. The wick may be made of any suitable absorbent material, such as hydrophilic polyethylene material, acrylic fibre, filter paper, or the like known in the art. The wick has the function of transferring the sample to the rest of the matrix for the assay. The sample receiving zone, or a portion of the sample receiving zone, may comprise a portion which is capable of changing colour on exposure to the sample. For example, this may be a chemical which changes colour on becoming moist. This may be used to ensure that the same device is not used for consecutive tests by mistake. A lid may also be provided for covering the sample receiving zone prior to use, or indeed after use, to prevent drips from the sample receiving zone.

The devices of the invention are preferably provided with a suitable housing to prevent accidental contact with the components making up the device. The housing may comprise one or more windows for observing the test zones and/or control zone. Where a wick is not used, the assay device may comprise an aperture or well for receiving the sample. The housing may also comprise one or more ridged or shaped portions to allow the device to be easily handled. The housing may be formed from a suitable plastics material of a type generally known in the art.

Preferably, the devices of the invention are provided in the form of kits comprising a plurality of devices. This allows the devices to be used on different days during the pregnancy, so that the concentrations between the different days of the pregnancy can be observed. Hence, a kit preferably comprises 2, 3 or more devices.

The devices may also be used in conjunction with conventional pregnancy detection kits which typically detect 25-40 mIU hCG.

A further aspect of the invention provides a method for monitoring a pregnancy comprising:

(i) providing a first sample and detecting a first concentration of a pregnancy associated hormone, using a device according to the invention;
(ii) providing a second sample at a later date than the first sample and detecting the concentration of a pregnancy associated hormone in the second sample using a device according to the invention; and
(iii) comparing the first concentration with the second concentration to obtain an indication of the progression of the pregnancy.

A still further aspect of the invention provides a method of monitoring a non-naturally induced termination comprising:

(i) providing a first sample and detecting a first concentration of a pregnancy associated hormone in the sample with a device according to the invention;
(ii) inducing a termination of the pregnancy, for example via a chemical induced abortion;
(iii) providing a second sample at a later date than the first sample and detecting the concentration of the pregnancy associated hormone in the second sample using a device according to the invention; and
(iv) comparing the first concentration with the second concentration to obtain an indication of the progression of the pregnancy.

The sample will usually be tested in vitro using, for example, a blood, saliva or most preferably urine sample.

Preferably, in the methods of the invention, the first concentration is recorded on the device, or packaging of the device, used to detect the concentration in the second sample.

The invention also provides a method of monitoring if a non-naturally induced termination has occurred comprising testing a sample from a patient who has had a termination induced, measuring a concentration of a pregnancy associated hormone with a device for detection and providing an indication of the concentration of a single pregnancy associated hormone (especially hCG), comprising a matrix defining an axial flow path, the matrix comprising:

(i) a sample receiving zone at the upstream end of the flow path, for receiving a fluid sample; and
(ii) a plurality of test zones positioned within the flow path and downstream from the sample receiving zone, wherein each test zone is capable of detecting a different concentration of hormone compared with an adjacent test zone; and
(iii) comparing the concentration of the hormone with a concentration from a sample before or immediately after the termination was induced.

Preferably the device, or its packaging, comprises an indicator for recording a previous hormone concentration recorded by the same or a different type of device.

The device may comprise one or more features defined for the device according to the invention.

The concentration taken before or immediately after the termination may be obtained using the same or a different type of assay device.

Preferably, a reduction in the concentration of the pregnancy-associated hormone indicates that an abortion or miscarriage has taken place. Such a change provides an indication that an abortion or miscarriage has taken place, so that the user knows whether to approach a doctor for confirmation that this has taken place.

Most preferably, the devices of the invention are hand-held and provide a visual indication of the concentration.

Preferably, consecutive tests are carried out at 1, 2, 3, 4, 5, 6, 7, 8, 9, 10, 11, 12, 13 or 14 day intervals.

The avidin/biotin system described above may also be adapted for conventional pregnancy test kits to improve their sensitivity.

A further aspect of the invention provides a device for the detection of a pregnancy associated hormone (especially hCG) comprising a matrix defining an axial flow path, the matrix comprising;

(i) a sample receiving zone at the upstream end of the flow path for receiving a fluid sample; and
(ii) one or more test zones positioned within the flow path and downstream of the sample receiving zone, said sample receiving zone comprising a restraint element comprising a first half of an avidin/biotin binding pair, the device additionally comprising:
(iii) an anti-pregnancy associated hormone antibody, such as an anti-hCG antibody, linked to a second half of an avidin/biotin binding pair.

The components of the device may be individually or together the same as those defined above for any aspect of the invention.

The device may simply detect whether a single concentration of hormone has been exceeded. This may be in between 25-40 mIU/ml.

The invention will now be described by way of example only, with reference to the following figures:

FIG. 1 shows typical hCG levels during normal pregnancy and spontaneous miscarriage or termination. The preferred concentrations of hCG detected (“test levels”) are also shown.

FIG. 2 shows the same data as FIG. 1, but on a logarithmic graph.

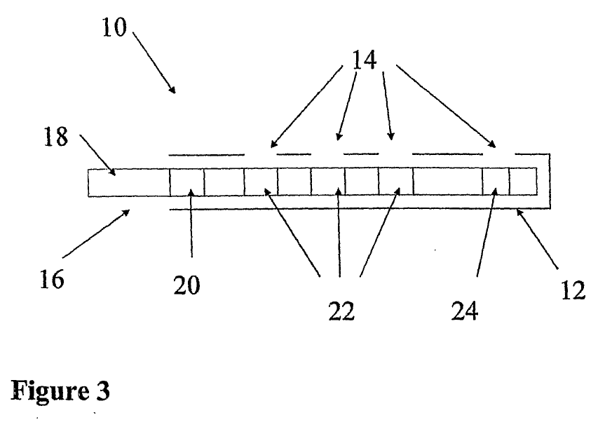

FIG. 3 shows a schematic cross-section through a device according to the invention.

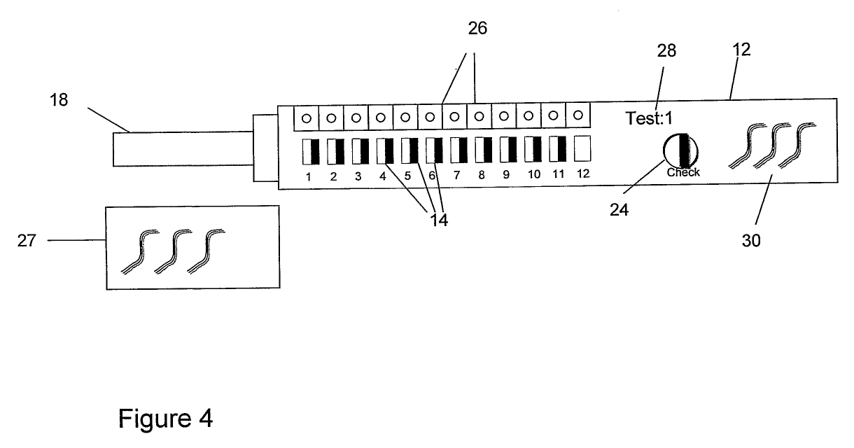

FIG. 4 shows a view of an assay kit from above.

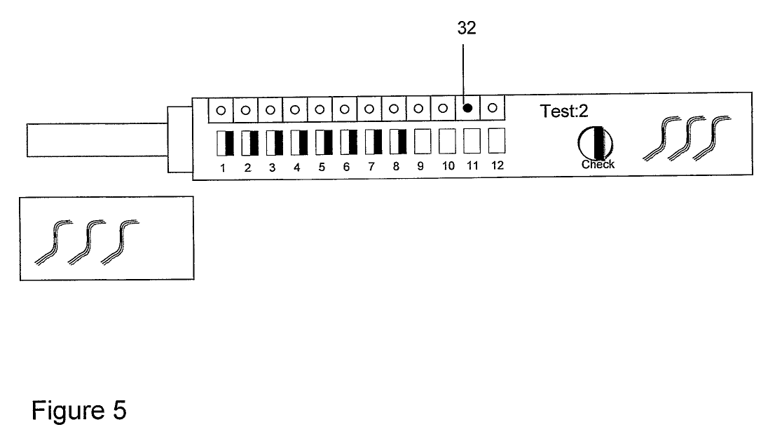

FIG. 5 shows a second assay kit from above.

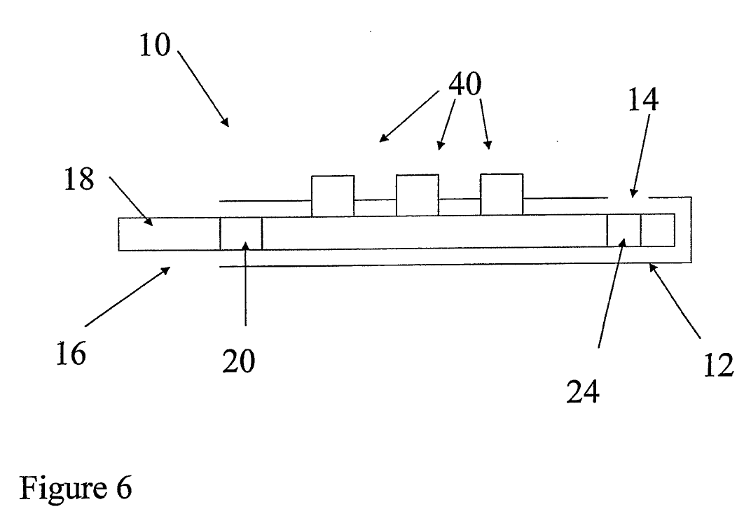

FIG. 6 shows an alternative device from one side with test zones extending out of the matrix.

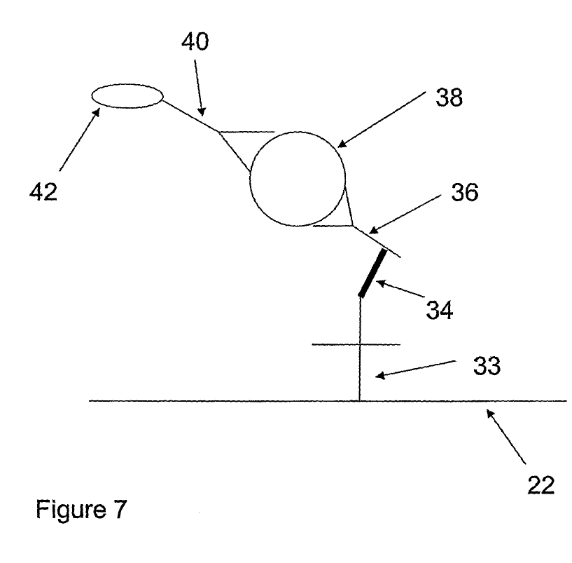

FIG. 7 shows a schematic representation of a preferred test zone arrangement.

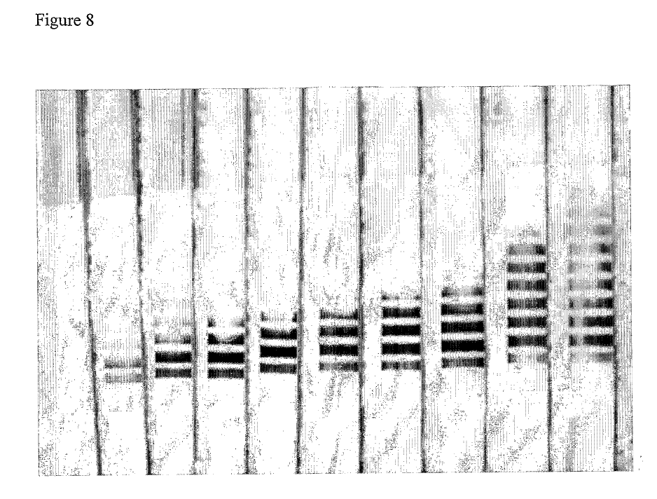

FIG. 8 shows the detection of different concentrations of hCG by example assay devices. Concentrations A 0, B 500, C 2500, D 7000, E 15000, F 27500, G 40000, H 55000, I 75000, J 100000 mIU hCG/ml.

We refer initially to FIG. 3. This shows an assay kit, 10, comprising a housing, 12. The housing comprises a plurality of windows, 14, allowing visual access to a matrix, 16. The matrix comprises a sample receiving zone, 18. The sample receiving zone, 18, comprises a wick for absorbing a liquid sample, for example a urine sample. The urine sample flows through the wick and through the matrix, 16. The wick may be any suitable absorbent material, such as paper, and preferably comprises a compound that changes colour upon contact with a liquid. The matrix itself may be any suitable material, as described above. Most preferably, it is a high density polyethylene sheet material. The fluid sample flows between the sample receiving zone, 18, and through a label zone, 20. The label zone provides a labelled reagent which is capable of binding the pregnancy associated hormones within the sample and is mobilisable in the presence of the fluid sample. The labelled reagent is preferably capable of binding hCG. Typically, the labelled reagent will be an anti-hCG antibody labelled with a visual label such as a gold sol or other coloured label. This results in the labelling of the pregnancy associated hormone, such as hCG as it passes through the matrix, 16. The labelled hormone then passes through a series of test zones, 22. The test zones comprise immobilised antibodies in different concentrations. The antibodies are specific for the pregnancy associated hormone. Typically, they will be goat or sheep anti-human chorionic gonadotrophin. These bind to and immobilise the coloured hormone. Lower concentrations of hormone are detected by the test zones proximal to the sample receiving zone, 18. Higher concentrations are detected in the test zones, 22, distal to the sample receiving zone, 18. Where sufficient concentration of labelled hormone passes through the matrix, 16, to the individual test zones, this results in a coloured band at the site of the test zone, 22. This coloured band will be visible through the windows, 14. If only a low concentration of hormone passes through so that only one of the test zones, 22, is coloured, the remaining, higher concentrations will not become coloured as most of the labelled hormone will have been trapped by the test zones proximal to the sample receiving zone, 18. At progressively higher concentrations, more test zones, 22, will be activated.

A control zone, 24, is provided. When sufficient sample has progressed through the matrix, 16, the sample will pass into the control zone, 24. The control zone, 24, comprises a suitable chemical that becomes coloured on application of moisture. This coloured band can be viewed through one of the windows, 14.

FIG. 4 shows a preferred embodiment of the invention. It utilises 12 test zones which are visible through windows, 14. The test zones measure: 40, 100, 400, 1600, 4000, 8000, 16000, 32000, 64000, 96000, 132000, 164000 mIU/ml. A test zone, 24, is visible through a window. Also visible are the wick which provides the sample receiving zone, 18, and a lid, 27, for covering the wick before and after use. The figure also shows an indicator or recordal zone, 26. This is for recording the concentration of hormone detected in a sample. In this particular case, the hormone is detected in 11 out of 12 of the windows. This correlates to a concentration of approximately 132000 to 164000 mIU/ml. This is test 1, as indicated by the label, 28. Hence the indicator has not been used.

The figure also indicates a series of ridges, 30, which are embossed into the casing, 12, and allow the device to be manipulated and handled more easily.

FIG. 5 shows a second test kit. In FIG. 5 it should be noted that the concentration from the first test, shown in FIG. 4, has been indicated on the indicator strip, 26, at point, 32. The second test indicates a decrease in hCG. That is, colour is only indicated in windows 1-8 (correlating to a concentration of approximately 32000 to 64000 mIU/ml). This decrease in hormone concentration indicates that there may be a problem with the pregnancy and indeed indicates a miscarriage. This indicates to the user that they should seek medical advice.

The time difference between the two assays will typically be 1-4 days.

FIG. 6 shows an alternative embodiment in which the test zones, 40, extend out of the plane of the matrix, 16. As discussed above, the test zones preferably contain antibodies specific for the pregnancy associated hormone. The test zones, 40, preferably comprise the same matrix material as that of the bulk of the matrix, 16. A portion of the sample preferably moves into the test zone by capillary action. A molecular valve, of a type known in the art, may be used to ensure movement of the sample in one direction only, away from the plane of the matrix, 16. The remaining sample continues along the plane of the matrix to subsequent test zones, 40, or the control zone, 24.

The advantage of this arrangement is that it helps prevent leaching of captured label from adjacent test zones.

FIG. 7 shows a preferred use of an avidin biotin binding pair. The test zone (22) comprises avidin (32) bound to its surface. In the figure hCG (38) has been trapped onto the avidin (33) via a monoclonal anti-hCG antibody (36) which is attached to a biotin molecule (34). The biotin (34) binds to the avidin (33) to restrain the hormone (38) at the test zone (22) via the antibody (36). The hormone (38) may be detected by the binding of a polyclonal antibody (46) labelled with, for example, a latex label (42). Alternatively, biotin may be attached to the test zone and the monoclonal anti-hCG antibody linked to avidin instead. The labelled polyclonal antibody (40, 42) and biotinylated antibody (34, 36) may be provided at the sample receiving zone or a label zone of the device where they are able to contain hormone in a sample placed at the zone, prior to moving through the matrix to the test zone.

FIG. 8 shows the detection of different concentrations of hCG by test strips.

### Materials & Reagents:

S&S FF60 nitrocellulose, pt# 10534315, lot# DH0348-1
Wick: Whatman 470 lot# F015
Conjugate pad: Whatman Standard 17 blocked with 10 mM sodium tetraborate, 4% goat serum, 3% BSA, 1% PVP, 0.25% triton X-100 and sprayed with OD50 mouse anti hCG-gold+1 mg/ml mouse IgG, (lot# 2/26/07MA)
Mouse anti hCG-Gold conjugate previously sprayed onto blocked Standard 17, lot: 031907MA
Membrane Blocking Buffer: 10 mM Phosphate, 0.1% BSA, 0.2% PVP, 4% sucrose, 0.1% azide, lot: 032107MA)
G&L 60 mm laminate backing
75×100 mm glass tubes for running test strips
Test lines: Goat anti hCG (Fitzgerald, lot# X0610190)
Control line: Goat anti mouse IgG (Quad Five lot # 41-4222)
HCG standard (Fitzgerald lot# 015K1453)
Female urine lot: 022807 (for diluting hHCG standards)
Biodot Frontline Instrument

### Methods

The 25 mm FF60 was laminated onto a 16 mm backing card. The control antibody, goat anti-mouse, was diluted to 0.5 mg/ml in PBS (phosphate buffered saline). The control antibody was then primed into a BioJet Quanti line No. 2 and dispensed using a Biojet dispenser at 1 μl/cm speed=50 mm/second. The control line was sprayed at 5 mm from the top of the nitrocellulose.

The test line antibody was then diluted, goat anti-hCG to 3, 2 and 1 mg/ml in PBS. 1 mg/ml of antibody solution was primed into the BioJet Quanti line No. 1 and dispensed using the BioJet dispenser at 1 μl/cm at a speed of 50 mm/sec. Test lines were sprayed 1.8 mm apart.

The line was then emptied and primed with 2 mg/ml antibody solution into the BioJet Quanti line No. 1 and dispensed using a Frontline dispenser at 1 μl/cm at a speed of 50 mm/sec. Spray test lines were as indicated in the table below.

This was repeated for the 3 mg/ml antibody solution and the card allowed to dry at 37° C. for 30 mins. The membrane was blocked using 10 ml of membrane block by slowly dragging the membrane through the blocking buffer and allowing it to wick through the material. The card was then placed onto a paper towel to blot any excess liquid from the top of the membrane and dried at 37° C. for 30 minutes.

The striped and dried conjugate pad was then cut to 20 ml and laminated onto the membrane with a 2 ml overlap. A wick was cut to 19 mm and laminated onto the membrane with a 2 mm overlap. The test card was then cut into 4 mm strips using a cutter.

To test the strips, 100 μl of hCG standard was pipetted into the bottom of a glass test tube. Strips were added with the conjugate pad down and the strips were allowed to absorb the test material through the strip. After 20 minutes the strips were removed from the tubes and photographed.

Numbers in table refer to mg/ml goat anti hCG striped on test line.

| Test Line | | | | | | | | | |
|---|---|---|---|---|---|---|---|---|---|
| 1 | 2 | 3 | 4 | 5 | 6 | 7 | 8 | 9 | Total |
| 1 | 3 | 2 | 2 | 2 | 2 | 2 | 2 | 2 | 18 |

### Results:

Concentrations (mIU hCG/ml)

### A—0 F—27,500

### B—500 G—40,000

### C—2500 H—55,000

### D—7000 I—75,000

### E—15000 J—100,000

The results are shown in FIG. 8.

1. A device for detection of and providing an indication of the concentration of a single pregnancy associated hormone, comprising a matrix defining an axial flow path, the matrix comprising:
(i) a sample receiving zone at the upstream end of the flow path, for receiving a fluid sample; and
(ii) a plurality of test zones positioned within the flow path and downstream from the sample receiving zone, wherein each test zone is capable of detecting a different concentration of hormone compared with an adjacent test zone; additionally comprising:
(iii) an indicator for recording a previous hormone concentration recorded by the same or a different device.

2. A device according to claim 1, wherein the indicator is on the device or on packaging for the device.

3. A device according to claim 1 or claim 2, which is disposable.

4. A device according to claim 1, which is adapted to detect a decrease in the pregnancy associated hormone associated with a miscarriage or non-natural associated termination of a pregnancy.

5. A device according to claim 1, wherein the device is a dipstick.

6. A device according to claim 1, wherein a label zone is positioned within the flow path, and between the sample receiving zone and plurality of test zones, said label zone comprising a labelled reagent which is capable of binding the pregnancy associated hormone and is mobilisable in the presence of a fluid sample, wherein said test zones each contain restraint elements which permit the restraint of labelled pregnancy associated hormone.

7. A device according to claim 6, wherein the restraint elements are antibodies, or fragments thereof, specific for the pregnancy associated hormone.

8. A device according to claim 6, wherein the restraint element comprises a first half of an avidin/biotin binding pair, and the device additionally comprises an anti-pregnancy associated hormone antibody linked to a second half of the avidin/biotin binding pair.

9. A device according to claim 1 wherein the concentration of pregnancy associated hormone capable of being detected increases between a test zone closest to the sample receiving zone and a test zone further away from, downstream of, the sample receiving zone,

10. A device according to claim 1 comprising 6 to 30 test zones.

11. A device according to claim 1 wherein said hormone is hCG and the concentration of hCG detectable by the device is between 10 to 260,000 mIU/ml sample.

12. A device according to claim 11, wherein the concentration of hCG detectable is between 150 and 165,000 mIU/ml.

13. A device according to claim 1 wherein the detection zones detect approximately the following concentrations of hCG: 400, 2500, 7000, 15000, 27500, 40000, 55000, 75000, 100000 mIU/ml.

14. A device according to claim 1, comprising a detection zone to detect a concentration of hCG of one or both of 1500-2500 mIU/ml and/or 4000-7000 mIU/ml,

15. A device according to claim 1 additionally comprising a control zone positioned within the flow path and downstream of the control means, and comprising means for permitting the indication of the completion of the assay.

16. A device according to claim 1, wherein the sample receiving zone comprises a wick.

17. A device according to claim 16, wherein the wick is capable of changing colour on exposure to a sample.

18. A device according to claim 1, additionally comprising a lid for covering the sample receiving zone.

19. A kit for following the progression of a pregnancy comprising a plurality of devices according to claim 1.

20. A method of monitoring a pregnancy comprising:
(i) providing a first sample and detecting a first concentration of a pregnancy associated hormone in the sample optionally with a device according to claim 1;
(ii) providing a second sample at a later date than the first sample, and detecting the concentration of the pregnancy associated hormone in the second sample using a device according to claim 1; and
(iii) comparing the first concentration with the second concentration to obtain an indication of the progression of the pregnancy.

21. A method of monitoring non-naturally induced termination comprising:
(i) providing a first sample and detecting a first concentration of a pregnancy associated hormone in the sample;
(ii) inducing a termination;
(iii) providing a second sample at a later date than the first sample, and detecting the concentration of the pregnancy associated hormone in the second sample using a device according to claim 1; and
(iv) comparing the first concentration with the second concentration to obtain an indication of the progression of the pregnancy.

22. A method of monitoring if a non-naturally induced termination has occurred comprising testing a sample from a patient who has had a termination induced, measuring a concentration of a pregnancy associated hormone with a device for detection and providing an indication of the concentration of a single pregnancy associated hormone, comprising a matrix defining an axial flow path, the matrix comprising:
(i) a sample receiving zone at the upstream end of the flow path, for receiving a fluid sample; and
(ii) a plurality of test zones positioned within the flow path and downstream from the sample receiving zone, wherein each test zone is capable of detecting a different concentration of hormone compared with an adjacent test zone; and
(iii) comparing the concentration of the hormone with a concentration from a sample before or immediately after the termination was induced.

23. A method according to any one of claims 20 to 22, wherein the first concentration is recorded on the device used to detect the concentration in the second sample, or its packaging.

24. A method according to claim 23, wherein a reduction in the concentration of the pregnancy-associated hormone indicates that an abortion has taken place.

25. The method according to claim 22, wherein said hormone is hCG.
